(19) United States
(12) Patent Application Publication
(10) Pub. No.: US 2008/0097229 A1
(43) Pub. Date: Apr. 24, 2008

(54) Hygroscopic treatment for degenerating discs

(76) Inventors: Josee Roy, Memphis, TN (US); Susan J. Drapeau, Cordova, TN (US); Jeffrey C. Marx, Germantown, TN (US); Sean M. Haddock, Memphis, TN (US)

Correspondence Address: FOX ROTHSCHILD, LLP, 997 LENOX DRIVE, LAWRENCEVILLE, NJ, 08648, US

(21) Appl. No.: 11/511,701

(22) Filed: Aug. 29, 2006

(51) Int. Cl. A61M 31/00; B65D 69/00

(52) U.S. Cl. 600/500; 206/223

(57) ABSTRACT

Early treatment for a degenerated disc includes identifying a non-ruptured, dehydrated disc. A hygroscopic agent is then selected. This agent should pull water towards itself and have a hygroscopic compound with a molecular weight of at least 400 Daltons. Additionally, it may be desirable to selected hygroscopic compounds that are anionic or electrically neutral. The hygroscopic agent is then injected into the nucleus pulposus of the disc without any prior removal of the nucleus pulposus material. Because the surrounding disc structure allows for the passive diffusion of water into the nucleus pulposus, the hygroscopic agent will draw water into the nucleus pulposus, thus promoting tissue hydration within the disc. The hygroscopic agent may also include a biological agent and/or an imaging agent.

### FIELD OF THE INVENTION

The present invention relates to methods for treating degenerative discs, and more specifically discloses utilizing hygroscopic materials to encourage hydration of a degenerative disc.

### BACKGROUND OF THE INVENTION

As shown in FIG. 1, the spine 10 is composed of a column of vertebrae 12 that are individually separated from each other by interposed discs 20. The spinal cord 14 runs through the length of the spine 10. The discs 20 are an important part of the entire spinal column 10, and act like shock absorbers between adjacent vertebrae 12. The discs 20 must be able to absorb mechanical loads while simultaneously permitting constrained flexing of the spine 10.

Each disc 20 is shaped somewhat like a jelly donut, having a relatively soft inner region 22 surrounded by a strong, fibrous outer region 24. The gel-like inner region 22 is called the nucleus pulposus, and the reinforcing outer region 24 is called the annulus fibrosis. The nucleus pulposus 22 distributes mechanical loads placed upon the disc 20, while the annulus fibrosis 24 provides structural integrity and constrains the nucleus pulposus 22 to a specific spinal region. Healthy discs 20 are typically avascular, and hence rely on diffusion through the annulus fibrosis 24 for the transport of nutrients and metabolites into and out of the nucleus pulposus 22. See generally Jill P. G. Urban et al., Nutrition of the Intervertebral Disc, 29 SPINE 2700-09 (Vol. 23, 2004).

Degenerated discs are a significant source of spine-related pain. Amongst sufferers of chronic pain, spine-related problems constitute the bulk of such complaints. Spinal pain has been estimated to exist in as much as 66% of the general population. Beyond the substantial discomfort that back pain inflicts upon individuals, spine-related pain also incurs heavy societal costs. For example, as many as one million spine surgeries, and as many as five million interventional procedures, are estimated to be performed in the United States each year. Well beyond the purely medical and psychological burdens imposed by such procedures, the subsequent social costs related to productivity, disability compensation and lost taxes are substantial.

Currently, one of the most common treatments for discogenic pain is the removal of the disc associated with the pain; the adjacent vertebrae are then fused together, using either bone grafts, mechanical implants, or a combination of the two. This is drastic, highly invasive surgery that is both expensive and problematic. In particular, recovery times range from six months to over a year, and as many as 50% of patients experience subsequent pain levels to a degree that is equal to, or even exceeds, their pre-surgery levels. Also, successful fusions of vertebrae result in reduced articulation of the spinal column.

Alternative methods exist for the treatment of discs that are not so far gone as to be unsalvageable. For example, United States Patent Application Publication No. 2002/0151979 to Lambrecht et al. entitled “Device and Methods for Nucleus Pulposus Augmentation and Retention” discloses ways of alleviating and preventing further herniation of the nucleus pulposus. United States Patent Application Publication No. 2005/0203206 to Hai H. Trieu entitled “In-Situ Formable Nucleus Pulposus Implant with Water Absorption and Swelling Capability” discloses replacement of the nucleus pulposus with a polymeric composition. Both of these methods, however, target discs that already exhibit significant symptoms of degeneration; that is, the treatment occurs rather late in the degenerative stage of the disc. Moreover, these are relatively complicated procedures involving the placement of carefully designed implants within the disc body.

It would therefore be desirable to provide a minimally invasive method for treating discs at a relatively early stage of degeneration. It would be particularly beneficial if the treatment method were easy to perform, and hence easy for doctors to learn and apply.

### SUMMARY OF THE INVENTION

The instant invention provides for relatively early treatment of a degenerated disc. In one embodiment, a non-ruptured disc is identified. In a specific embodiment, the disc may also exhibit symptoms of dehydration. A non-ruptured disc is a disc that does not exhibit any release of nucleus pulposus material as revealed by imaging techniques. Non-ruptured discs may also be identified during discography.

A hygroscopic agent is then selected. This agent should have a hygroscopic compound that pulls water towards itself, and which should have a composition that resists passage through the annulus fibrosis, such as due to molecular charge, weight, viscosity of the formulation or the like. In a specific embodiment, the hygroscopic compound has a molecular weight of at least 400 Daltons, and is ideally anionic or electrically neutral. The hygroscopic agent is then injected into the disc without any prior removal of nucleus pulposus material. In a specific embodiment, the hygroscopic agent is injected into the nucleus pulposus. Because the surrounding disc structure allows for the passive diffusion of water into the nucleus pulposus, the hygroscopic agent will draw water into the nucleus pulposus, thus promoting tissue hydration within the disc.

In a specific embodiment, the hygroscopic agent may be biodegradable. More specifically, the hygroscopic agent may be a biodegradable polymer, such as polyethylene glycol.

### BRIEF DESCRIPTION OF THE DRAWINGS

FIG. 1 is a perspective view of a portion of a spinal column.

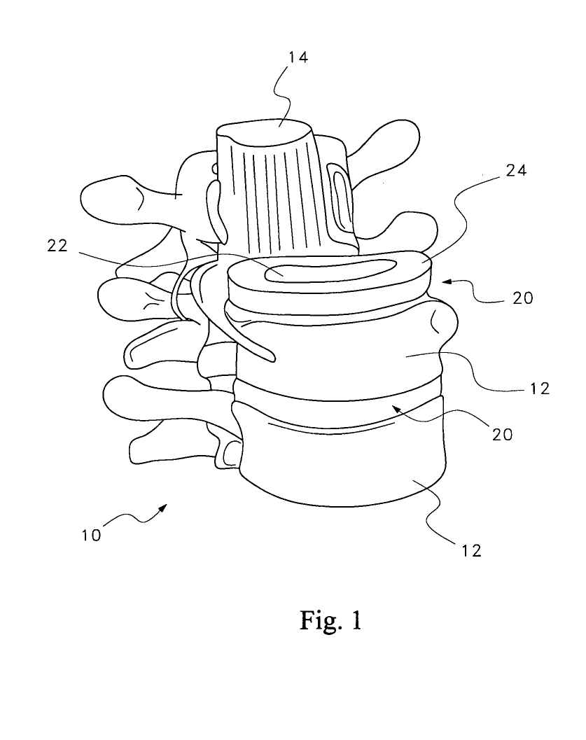

FIG. 2 is a side view of a spinal column illustrating various types of degenerated discs.

FIG. 3 is a side view illustrating treatment of a disc.

FIG. 4 is a cutaway perspective view of the treatment performed in FIG. 3.

### DETAILED DESCRIPTION

The terms “include,” “comprise,” “for example” or the like are intended in the following to be inclusive, rather than limiting. Hence, when presented, and unless indicated otherwise, lists of compounds, substances, or the like present merely exemplary subsets of potentially much larger sets. Items not listed but having characteristics known to be compatible in the context of those listed should also be understood to be within the ambit of the instant disclosure.

Used herein, “hygroscopic” indicates a substance that has an affinity for water. The term “hygroscopic” as used herein is interchangeable with the term “hydroscopic.” A hygroscopic agent is a therapeutic composition comprising a hygroscopic compound and designed to at least attract water to itself. That is, the hygroscopic compound should readily absorb water from its surroundings. As known in the art, the moisture content of a product can be defined as the percentage weight of water in relation to the dry weight of the product. Products in which moisture can be present can be classified in two categories: hygroscopic and non-hygroscopic. Examples of hygroscopic materials are salts, vegetal fibers, many polymers, etc. Examples of non-hygroscopic products are metal powders, glass granules, etc. Regarding the moisture content of a product, the static equilibrium can be defined as a set of conditions under which the product does not exchange any moisture with its environment. Under conditions of static equilibrium, the moisture content of a hygroscopic product depends on the nature of the product and also on the two following factors: (a) the partial pressure of water vapor in the immediate environment of the product; and (b) the temperature of the product. If the moisture content of a product is not dependent on both these factors, then the product is not hygroscopic.

In the following, a “hygroscopic compound” is any substance, or combination of substances, that exhibits hygroscopic properties, and which is selected primarily for those hygroscopic properties to impart such hygroscopic properties to the hygroscopic agent. The hydroscopic compound is ideally biocompatible. The degree of hygroscopicity a compound exhibits may be classified into one of four classes (see J. C. Callahan et al., Equilibrium Moisture Content of Pharmaceutical Excipients, 8 DRUG DEVELOPMENT AND INDUSTRIAL PHARMACY 355-69 (1982)): 1. non-hygroscopic solids—no increase in water content at <90% RH (relative humidity), and increase after storage for one week above 90% RH is less than 20%. 2. slightly hygroscopic solids—no moisture increase at <80% RH and increase after one week storage less than 40% at >80% RH. 3. moderately hygroscopic solids—moisture increase≦5% after storage at below 60% RH, and after storage for one week at >80% RH is less than 50% moisture. 4. very hygroscopic solids—moisture content increase may occur at 40-50% RH and increase may exceed 30% after storage for one week>90% RH.

In one embodiment, the contemplated hygroscopic agent comprises a slightly to very hygroscopic compound. In another embodiment the hygroscopic compound is part of a liquid or semisolid injectable formulation. In another embodiment, the injected liquid formulation forms a semi-solid or gel-like material following injection into the disc. For example, materials that are liquid at room temperature, but which become gel-like at body temperature are known in the art. A hygroscopic agent may contain such materials to cause the hygroscopic agent to form a gel when injected into the patient. Alternatively, an appropriate combination of polymers, as known in the art, may form a gel when injected into a patient, and may additionally have hygroscopic properties suitable for the instant invention method.

A hygroscopic agent will include at least one hygroscopic compound, alone or with diluents, excipients, imaging agents, biologically active agents or other pharmaceutically acceptable ingredients desirable for improved stability, manufacturing, efficacy and the like, or which facilitate delivery of the agent to the disc, evaluate retention of the agent within the disc or the like.

An “imaging agent” is defined as a substance added to the hygroscopic agent and that can be visualized by imaging techniques including radiography, MRI, PET or SPECT, CT, fluoroscopy, luminescence and any combination thereof. Imaging agents can be used to facilitate optimal delivery of the hygroscopic agent to the disc. The imaging agents may also allow for evaluation of the retention of the hygroscopic agent within the disc over time.

Suitable non-limiting examples of an imaging agent may be a radiographic marker, such as for example, barium, calcium phosphate, and/or metal beads. In another embodiment, the imaging agent may comprise iodine-based contrast agents, such as, for example, iopamidol, commercially available as Isovue™ (Bracco Diagnostics Inc., Princeton, N.J.) or iodixanol, commercially available as Visipaque™ (Nyocomed, Inc., Princeton, N.J.), and gandolinium-based contrast agents, such as, for example, gadodiaminde, commercially available as Omniscan™ (available from GE Healthcare, Princeton, N.J.).

Other examples would include linkage of the imaging agent or radioisotope to a component of the hygroscopic agent. Examples of radioisotope would include ¹⁸F, ³H, ¹²⁴I, ¹²⁵I, ¹³¹I, ³⁵S, ¹⁴C, ¹¹C or a fluorescent molecule.

Radioisotopes may be attached to a component of the hygroscopic agent, such as the hygroscopic compound, by using a chelating agent, such as EDTA or DTPA, and detected by gamma counter, scintillation counter, PET scanning, or autoradiography. Other methods of labeling the marker are described, for example, in the U.S. Pat. App. No. 2005/0118165 and in Hunter et al., Nature 194:495 (1962); G. S. David et al., Biochemistry 13:1014-1021 (1974); D. Pain et al., J Immunol Meth 40:219-230 (1981); and H. Nygren, J. Histochem Cytochem. 30:407 (1982), all of which are incorporated by reference herein.

In other embodiments, the imaging agent is a fluorescent label. Common fluorescent labels include fluorescein, dansyl, phycoerythryn, phycocyanin, allophycocyanin, o-phtaldehyde, and fluorescamine. In yet other embodiments, the imaging agent may comprise a fluorescence-emitting metal such as, for example, ¹⁵²Eu⁺ or other lantanoids. The fluorescence-emitting metals can be attached to a component of the hygroscopic agent, such as the hygroscopic compound, by using metal-chelating groups such as EDTA or DTPA.

In another embodiment, since radioisotopes may have a limited half-life, the imaging agent may be added to the hygroscopic agent within a few hours prior to administration.

When employed, the amount of excipient that is useful in the hygroscopic agent is an amount that serves to uniformly distribute the hygroscopic compound and zero or more biologically active ingredients and zero or more imaging agents throughout the hygroscopic agent so that the hygroscopic compound and optional biologically active ingredients and imaging agents are uniformly dispersed when delivered to a subject in need thereof. The excipient may serve to dilute the hygroscopic compound, biologically active ingredients and imaging agent to a concentration at which the desired beneficial, palliative or curative results are obtained, while at the same time minimizing any adverse side effects that might otherwise occur from excessively high concentrations. The excipient may also have a preservative effect.

A “biologically active agent” is defined as an agent designed to achieve a medically useful end. In the context of this application, the biologically active agent would be used to treat signs of disc degeneration and pain.

In one aspect of the invention, a method for treating a degenerated disc includes administering an effective amount of a hygroscopic agent to the disc, in which the hygroscopic agent includes both a hygroscopic compound and a biologically active agent that is adapted to disrupt neuronal, vascular or immune elements in the disc, or in a region around the disc.

Examples of such biologically active agents include, but are not limited to, natural neurotoxins; neurotoxins comprising ammonia or cyanide; bisbenzimide; trypan blue; brilliant blue; methylene blue; indocyanine green; ruthenium red; quinoline yellow; saporin; Rho kinase activators; camphor; menthol; piperine; mustard oil; eugenol; curcumin; 8-Methyl-N-vanillyl-trans-6-nonenamide (Capsaicin); Z-Capsaicin; Gingerol; Zingerone; 8-Methyl-N-vanillylnonanamide (Dihydrocapsaicin); 6,7-Deepoxy-6,7-didehydro-5-deoxy-21-dephenyl-21-(phenylmethyl)-daphnetoxin,20-(4-hydroxy-5-iodo-3-methoxybenzeneacetate) (5′-Iodoresiniferatoxin)-?; (+)-Isovelleral; N-Vannilyloleoylamide (Olvanil); Phorbol 12,13-dinonanoate 20-homovanillate; Resiniferatoxin; N-(3-Methoxyphenyl)-4-chlorocinnamide (SB-366791); 2,3,4-Trihydroxy-6-methyl-5-[(2E,6E)-3,7,11-trimethyl-2,6,10-dodecatrienyl]benzaldehyde (Scutigeral); 6,7-Deepoxy-6,7-didehydro-5-deoxy-21-dephenyl-21-(phenylmethyl)-20-(4-hydroxybenzeneacetate)daphnetoxin (Tinyatoxin); capsaicin synthetics; capsaicin derivatives; botulinum toxin; anti-convulsants; anesthetics; analgesics; opioids; cannabinoids; N-[2-(4-Chlorophenyl)ethyl]-1,3,4,5-tetrahydro-7,8-dihydroxy-2H-2-benzazepine-2-carbothioamide (Capsazepine); [N-(4-Hydroxy-3-methoxyphenyl)methyl]-5Z,8Z,11Z,14Z-eicosatetraenamide] (Arvanil); N-(3-Methoxyphenyl)-4-chlorocinnamide (SB-366791); 5′-iodoresiniferatoxin; steroids; nonsteroidal anti-inflammatory compounds; COX inhibitors; modulators of TNF-alpha or IL-1 cytokines or receptors; NFkB modulators; minocyclin and fluorocitrate. These therapeutic compounds, formulations, and the methods of use for treatment of discogenic pain are described in detail in co-pending U.S. patent application Ser. No. 11/414,689, entitled “Biological Removal Of Vascular And/Or Neuronal Extensions From A Degenerating Disc,” filed on Apr. 28, 2006, which is incorporated into the instant disclosure by reference in its entirety.

Other examples of biologically active agents include anti-viral and anti-retroviral compounds, therapeutic proteins or peptides, therapeutic nucleic acids, cells, and gene therapy vectoring systems.

Particular examples of biologically active agents that may be employed with the hygroscopic agent are anti-inflammatory agents. Suitable anti-inflammatory agents include but are not limited to soluble tumor necrosis factor α receptors, monoclonal antibodies, polyclonal antibodies, antibody fragments, COX-2 inhibitors, metalloprotease inhibitors (such as TAPI), glutamate antagonists, glial cell derived neurotrophic factors, B2 receptor antagonists, Substance P receptor (NK1) antagonists, Downstream regulatory element antagonistic modulator (DREAM), iNOS, inhibitors of tetrodotoxin (TTX)-resistant Na+-channel receptor subtypes PN3 and SNS2, inhibitors of interleukins, TNF binding protein, dominant-negative TNF variants, Nanobodies™, kinase inhibitors (such as Gleevec, Herceptin, Iressa, imatinib (STI571), herbimycin A, tyrphostin 47, erbstatin, genistein, staurosporine, PD98059, SB203580, CNI-1493, VX-50/702, SB203580, BIRB 796, Glaxo P38 MAP Kinase inhibitor, RWJ67657, UO126, Gd, SCIO-469, RO3201195, and Semipimod), and combinations thereof.

Other suitable anti-inflammatory agents include but are not limited to Adalimumab, Infliximab, Etanercept, Pegsunercept (PEG sTNF-R1), Onercept, Kineret®, sTNF-R1, CDP-870, CDP-571, CNI-1493, RDP58, ISIS 104838, 1→3-β-D-glucans, Remicade, Lenercept, PEG-sTNFRII Fc Mutein, D2E7, Afelimomab, AMG 108, 6-methoxy-2-napthylacetic acid) or betamethasone, capsaiein, civanide, TNFRc, ISIS2302 and GI 129471, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibody (daclizumab, basilicimab), ABX (anti IL-8 antibody), recombinant human IL-10, HuMax IL-15 (anti-IL 15 antibody) and combinations thereof.

Still other anti-inflammatory agents include but are not intended to be limited to NF Kappa B inhibitors, including glucocorticoids such as clonidine, nonsteroidal anti-inflammatory drugs (NSAIDs), such as sulindac and tepoxalin, antioxidants, such as dithiocarbamate, and other compounds such as sulfasalazine [2-hydroxy-5-[-4-[C2-pyridinylamino)sulfonyl]azo]benzoic acid] and flucinolone.

If used, the type, concentration and amount of the biologically active agent within the hygroscopic agent will necessarily depend on the medical characteristics of the patient to whom the hygroscopic agent is being administered.

The hygroscopic agent includes at least one hygroscopic compound that is preferably selected to avoid diffusion through the annulus fibrosis of the disc so as to provide for better retention of the hygroscopic compound within the disc. When the hygroscopic agent is injected into nucleus pulposus, the majority of the hygroscopic compound should remain in the nucleus pulposus; some of the hygroscopic compound may diffuse into the annulus fibrosis, but substantially none of the hygroscopic compound should diffuse out of the disc itself. The annulus fibrosis of discs tend to more readily permit diffusion of cationic compounds over anionic or electrically neutral compounds; diffusion is also limited by molecular weight, with compounds having a molecular weight of 200 Daltons or less diffusing more readily than heavier compounds. With these two considerations in mind, molecular weight and molecular charge, the hygroscopic compound preferably has a molecular weight of at least 200 Daltons and is electrically neutral or anionic. More preferably, the hygroscopic compound has a weight of at least 400 Daltons and is anionic. Polymers may also be preferred over monomers. Multi-arm polymers may also be preferred over linear polymers.

The present invention provides an early interventional procedure that is intended to be minimally invasive. It may therefore be desirable to further select hygroscopic compounds that are biodegradable or bio-absorbable. Preferred hygroscopic compounds are therefore biodegradable/bio-absorbable compounds having a molecular weight of at least 400 Daltons, and which are electrically neutral or, preferably, anionic. Even more preferably, the hygroscopic compound is a polymer. Multi-arm polymers may also be preferred over linear polymers.

Hygroscopic compounds suitable for use in a hygroscopic agent include polymers such as poly (ethylene glycol) (PEG), a block copolymer containing a polyalkylene glycol, triblock containing a polyalkylene glycol, a block copolymer containing a polyalkylene oxide, triblock containing a polyalkylene oxide, polyvinyl alcohol, polyvinyl pyrrolidone, dextrans, poloxamine, pluronic polyols, dimethylsulfoxide, sodium carboxymethyl cellulose, poly(polyethylene glycol methacryalte), poly(glycerol methacrylate), poly(glycerol acrylatete), poly(polyethylene glycol acrylate), poly(alkyl oxazoline), phosphoryl choline polymers, sodium and potassium polymethacrylate, sodium and potassium polyacrylate, polymethacrylatic acid and polyacrylic acid, and combinations thereof. Although classified as slightly hygroscopic, PEG may be particularly suited for use as the hygroscopic compound in a hygroscopic agent.

Hygroscopic compounds suitable for use in a hygroscopic agent also include sugars and proteins, such as cellulose, starch, glycerol, glycerin, proteoglycans, hyaluronic acids and derivatives thereof. In one embodiment, the protein has a fibrous structure, such as collagen. In another embodiment, the hygroscopic agent is a glycoprotein, such as proteoglycans, that have a core protein and at least one and up to hundreds of carbohydrate chains, the so-called glycosaminoglycans.

In another embodiment, a biologically active agent is linked to a hygroscopic compound to increase the retention of the biologically active agent within the disc. Such hygroscopic compounds include poly (ethylene glycol), a block copolymer containing a polyalkylene glycol, triblock containing a polyalkylene glycol, a block copolymer containing a polyalkylene oxide, triblock containing a polyalkylene oxide, polyvinyl alcohol, polyvinyl pyrrolidone, dextrans, poloxamine, pluronic polyols, dimethylsulfoxide, starch, hydroxyethylstarch, sodium carboxymethyl cellulose, poly(polyethylene glycol methacryalte), poly(glycerol methacrylate), poly(glycerol acrylatete), poly(polyethylene glycol acrylate), poly(alkyl oxazoline), phosphoryl choline polymers, sodium and potassium polymethacrylate, sodium and potassium polyacrylate, polymethacrylatic acid and polyacrylic acid, glycerol, glycerin, sugars, hyaluronic acid, collagen, proteoglycans, and combinations thereof.

Linkage of the biologically active agent to the hygroscopic compound is a process well within the means of one having ordinary skill in the art, and may include, for example, amide, imide, imine, ester, anhydride, acetal, carbamate, carbonate, carbonate ester, disulphide groups, glutaraldehyde, pegylation protocols, etc. Similarly, to ensure that the imaging agent, if present, is retained for as long as possible within the disc, it may also be desirable to link the imaging agent to the hygroscopic compound, utilizing any suitable method to do so.

Early treatment for a disc may be provided using a suitably formulated hygroscopic agent based upon the above disclosure. In particular, non-ruptured discs that are beginning to show signs of dehydration may be targeted. For purposes of the following, a “non-ruptured” disc is one in which the annulus fibrosis is sufficiently intact so as to prevent the release of nucleus pulposus material from the disc. Signs of a rupture in the disc can be observed by known imaging techniques. In some cases, the imaging protocol includes the administration of a contrasting agent, as for example, in a discography. During discography, retention of the injected dye within the disc is indicative of a non-ruptured disc. While discography is a diagnostic procedure, there are both visualization and evocative components. The procedure commonly entails an initial application of local anesthetic (i.e. lidocaine, etc.) to the skin and/or subcutaneous tissue of the injection site. Then, an injection of 1-2 cc of contrast media (i.e. Omnipaque™ or similar) via a 22 gage spinal needle is placed percutaneously, often in a posteriolateral orientation. Needle placement into the nucleus pulposus and the path of the media is visualized under fluoroscopic imaging, and degree of disc degeneration as well as the presence of annular fissures is readily apparent. Furthermore, patient reaction to the increased pressure from the injection is also witnessed; the physician is attempting to recreate the ‘typical’ pain the patient experiences. If the discogram is positive, the pain-generating disc level is then identified for further treatment. It will be appreciated that the same methods employed to perform a discography may also be employed to inject a hydroscopic agent into the disc to effect hydration of the disc according to the present invention.

A degenerated disc may, but not necessarily always, show anatomical signs of degeneration, which can include changes in the height of the disc, the level of hydration of the disc, the presence of osteophytes or herniation. A reduction in the height of the disc may be one of the most common, early and easily detectable changes present in a degenerated disc. Another sign of degeneration is normally a loss of the T2 weighted signal on an MRI scan; this is indicative of a loss of hydration of the nuclear tissue. The disc may be any disc within a spinal column, including cervical, thoracic and lumbar discs. The degenerated disc can be a contained disc that simply shows signs of thinning or dehydration, or a herniated disc. Herniation could be of a contained nature called, for example, bulging of the disc. A herniated disc can also be ruptured with release of discal elements, such as the nucleus pulposus outside the disc, but such a disc may not be suitable for the instant treatment.

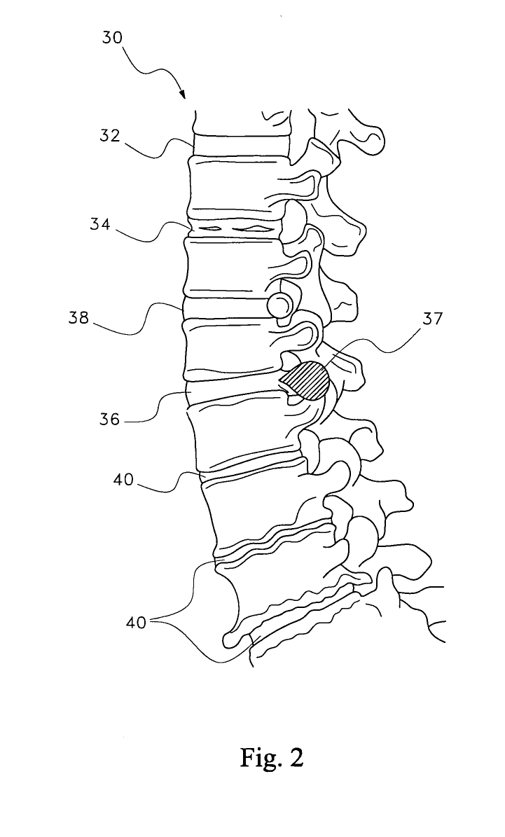

FIG. 2 shows a spinal column 30 with discs exhibiting different symptoms and degrees of degeneration. For reference, a healthy disc 32 is also shown. Degeneration of a disc can include rupturing as shown by discs 34 and 36. Disc 34 exhibits rupturing by way of tears in the annulus fibrosus. Disc 36 shows signs of ruptured herniation, in which the nucleus pulposus material 37 has extruded past the annulus fibrosus and extends externally from the disc 36; the annulus fibrosus has ruptured, thus permitting release of the nucleus pulposus. Contained herniation is shown by disc 38. In disc 38, herniation of the nucleus pulposus material creates bulging of the annulus fibrosis; no nucleus pulposus material, however, escapes from the confines of the annulus fibrosis. Disc 38 is thus a non-ruptured, herniated disc; the annulus fibrosis of disc 38 does not exhibit tearing, rupturing or the like. Discs 40 shows signs of thinning, but otherwise no signs of rupturing as there are no tears in the annulus fibrosis of the discs 40, nor is there any leakage of nucleus pulposus material from the discs 40. It is common to have disc height losses with disc degeneration. The inclusion criteria for patients with DDD, non-ruptured disc and being candidates for IDET, for example, would be similar to those patients identified for this therapy. Even so, the degree of height loss may vary with practitioners. Some more conservative physicians only treat discs with small amounts of height loss (i.e. 10-20% compared to adjacent levels), whereas others will include patients with disc height losses up to 50%. Height losses are often an accessory measure and not a threshold; such data is used in conjunction with other diagnostic information to identify not only which disc is symptomatic, but if such symptoms are appropriate for a specific therapy.

Once the target disc has been identified, and a hygroscopic agent formulated and obtained, a suitable amount of the hygroscopic agent is injected into the disc. For purposes of the following, the term “injected” is intended to mean that the hygroscopic agent is introduced into the disc, by whatever means; typically, the hydroscopic agent may be injected by way of a hypodermic needle, a catheter or the like, as described above with respect to discographies. Although the amount of agent actually injected into the disc will vary depending upon the specific characteristics of the disc, typically anywhere from 0.1 to 5 milliters may be injected. As indicated, the injection may be performed using a hypodermic needle of suitable length and gauge. The hygroscopic agent is preferentially injected wholly within the nucleus pulposus of the target disc, although it may also be at least partially injected within the annulus fibrosus. No nucleus pulposus material is removed from the target disc prior to the injection of the hygroscopic agent. That is, once the target disc is identified, the hygroscopic agent is thereafter administered to the target disc without any intervening treatment involving removal of nucleus pulposus material; the native nucleus pulposus material is simply augmented with the hygroscopic agent.

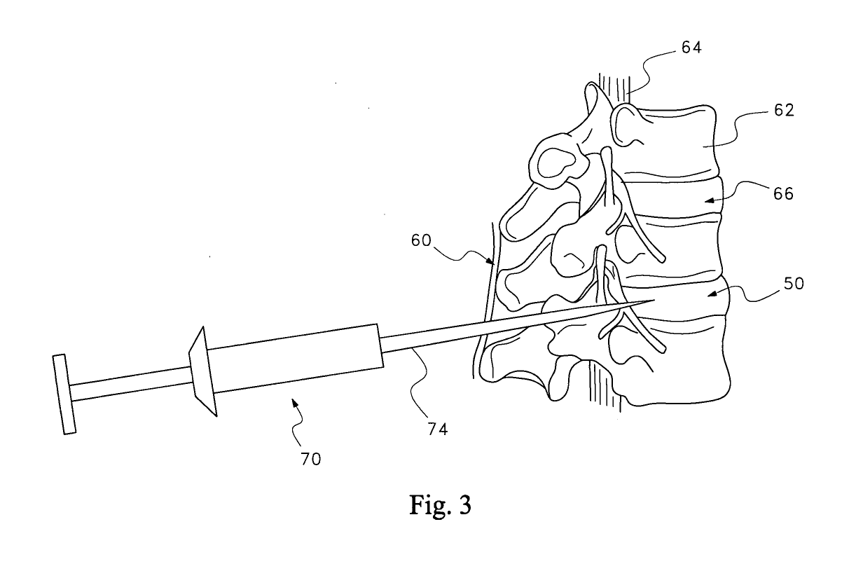

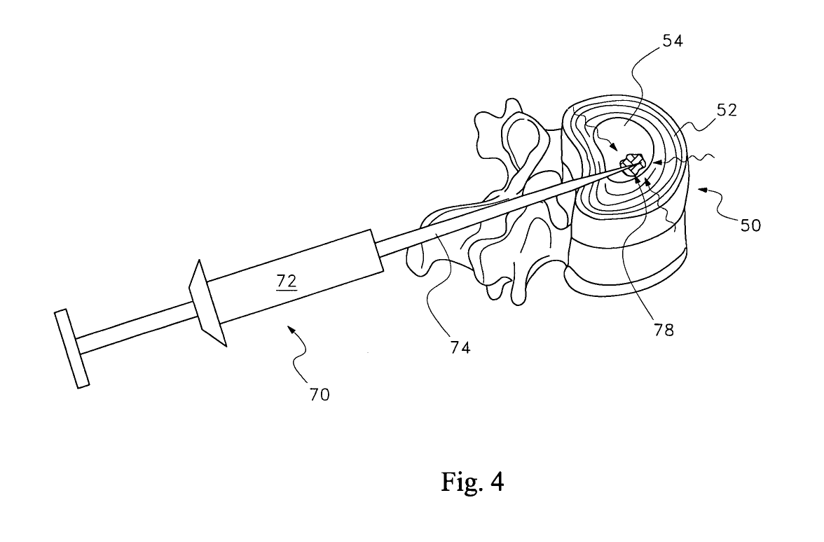

By way of example shown in FIGS. 3 and 4, a spinal cord 64 runs along the length of a spinal column formed from an alternating column of vertebrae 62 and discs 66. A specific non-ruptured disc 50 is identified as needing interventional treatment, perhaps due to dehydration. A syringe 70 loaded with a suitable amount of hygroscopic agent 72 and a hypodermic needle 74 is used to inject the hygroscopic agent 72 into the target disc 50. The hygroscopic agent 72 may thus be a liquid-phase material, albeit potentially very viscous, or may become gel-like after injection into the patient. After piercing the skin 60, a doctor carefully guides the hypodermic needle 74 towards the target disc 50. The needle 74 pierces through the annulus fibrosis 52 and terminates within the nucleus pulposus 54. The hygroscopic agent 72 is then injected through the cannula of the hypodermic needle 74 to leave a bolus 78 of hygroscopic agent 72 within the nucleus pulposus 54. Because of the molecular characteristics of the hygroscopic compound, the hygroscopic compound within the hygroscopic agent 78 is largely unable to diffuse out of the nucleus pulposus 54 into the annulus fibrosis 52, and substantially none of the hygroscopic compound diffuses out of the target disc 50. Note, however, that other bioactive agents within the bolus 78 may be able to diffuse into the annulus fibrosis 52, or even out of the disc 50, depending upon their respective molecular characteristics. The hygroscopic agent 78 has a high affinity for water which will, in effect, create a hydrostatic pressure that causes water to diffuse through the annulus fibrosis 52 into the nucleus pulposus 54, as indicated by the undulating arrows, thus helping to re-hydrate the disc 50.

It may be desirable to have the hygroscopic agent 78 disperse as widely as possible throughout the nucleus pulposus 54. One method to achieve such a dispersion is to perform several injections into the disc 50, coming in from different angles and penetration depths. However, such repeated puncturing of the annulus fibrosis 52 may be counter-productive to the desired results of treating the disc 50. An alternative method that may be used instead is disclosed in U.S. patent application Ser. No. 11/118,125 to Drapeau and McKay, entitled “Devices and Methods of Injecting Disc with Therapeutic Agents,” which is incorporated herein by reference. Briefly, a guide cannula may be inserted into the disc, and then two or more smaller dispersing cannulas may be threaded down the guide cannula; these smaller cannulas branch away from the guide cannula upon exiting the distal end, and thus cover a broader area between them than a single cannula. Additionally, these dispersing cannulas are equipped with apertures located periodically along their lengths, so that the hygroscopic agent is released not just at the distal ends, but at positions along the lengths of the dispersing cannulas as well. Alternatively, a cannula may be inserted into the disc that has contained within it several extensions. These extensions may be made of shape memory metal, such that when inserted into tissue at body temperature, they curve in specific directions designed to extend across much of the disc area. There may be two or more extensions in the device. These extensions have injection ports at the end or dispersed along the edge of the extensions. Fluid or small devices can be ejected from the ports.

To further provide for ease of use of the instant invention, it may be desirable to provide a kit that contains most or all of the elements required to practice the invention. For example, one embodiment for a kit may include a vial containing the hygroscopic agent in liquid form, a syringe and one or more needles for the syringe, all contained within sterile packaging. Or, the kit may contain the hygroscopic compound in dry or liquid form within one container, and an imaging agent in another container. The kit may optionally include one or more biologically active agents, either separately or admixed with the hygroscopic compound. The kit may also contain a suitable excipient or diluent which may be used as a solvent into which may be mixed suitable amounts of the hygroscopic compound, and optionally one or more biologically active agent or imaging agent. The kit should also include instructions describing how to administer the hygroscopic agent to a patient to treat a disc, and optionally how to mix together the hygroscopic compound, biologically active agent and/or imaging agent to form a suitable hygroscopic agent adapted for injection into a disc. The instructions may also indicate how to modify the hygroscopic agent for imaging purposes.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A method for treating a degenerated disc comprising:
identifying a non-ruptured disc; and
without prior removal of any nucleus pulposus material from the non-ruptured disc after identifying the non-ruptured disc, injecting a hygroscopic agent comprising a hygroscopic compound into the non-ruptured disc.

2. The method of claim 1 wherein the hygroscopic compound has a molecular weight of at least 400 Daltons.

3. The method of claim 1 wherein the hygroscopic agent is biodegradable.

4. The method of claim 1 wherein the hygroscopic compound comprises a polymer.

5. The method of claim 4 wherein the polymer is selected from a set consisting of poly (ethylene glycol), a block copolymer containing a polyalkylene glycol, triblock containing a polyalkylene glycol, a block copolymer containing a polyalkylene oxide, triblock containing a polyalkylene oxide, polyvinyl alcohol, polyvinyl pyrrolidone, dextrans, poloxamine, pluronic polyols, poly(polyethylene glycol methacryalte), poly(glycerol methacrylate), poly(glycerol acrylatete), poly(polyethylene glycol acrylate), poly(alkyl oxazoline), phosphoryl choline polymers, sodium and potassium polymethacrylate, sodium and potassium polyacrylate and polymethacrylatic acid, and polyacrylic acid.

6. The method of claim 1 wherein the hygroscopic compound comprises poly (ethylene glycol).

7. The method of claim 1 wherein the hygroscopic compound comprises a sugar.

8. The method of claim 7 wherein the sugar is selected from a set consisting of cellulose, starch, glycerol, glycerin, and derivatives thereof.

9. The method of claim 1 wherein the hygroscopic compound comprises a protein or glycoprotein.

10. The method of claim 9 wherein the protein is selected from a group consisting of proteoglycan, hyaluronic acid, collagen and derivatives thereof.

11. The method of claim 1 wherein the hygroscopic compound is anionic.

12. The method of claim 1 wherein the hygroscopic compound is electrically neutral.

13. The method of claim 1 wherein identifying the non-ruptured disc further comprises identifying symptoms of dehydration in the disc.

14. The method of claim 1 wherein the hygroscopic agent further comprises a biologically active agent.

15. The method of claim 14 wherein the biologically active agent is selected from a set consisting of natural or synthetic neurotoxins comprising ammonia or cyanide; bisbenzimide; trypan blue; brilliant blue; methylene blue; indocyanine green; ruthenium red; quinoline yellow; saporin; Rho kinase activators; camphor; menthol; piperine; mustard oil; eugenol; curcumin; 8-Methyl-N-vanillyl-trans-6-nonenamide (Capsaicin); Z-Capsaicin; Gingerol; Zingerone; 8-Methyl-N-vanillylnonanamide (Dihydrocapsaicin); 6,7-Deepoxy-6,7-didehydro-5-deoxy-21-dephenyl-21 (phenylmethyl)-daphnetoxin,20-(4-hydroxy-5-iodo-3-methoxybenzeneacetate) (5′-Iodoresiniferatoxin); (+)-Isovelleral; N-Vannilyloleoylamide (Olvanil); Phorbol 12,13-dinonanoate 20-homovanillate; Resiniferatoxin; N-(3-Methoxyphenyl)-4-chlorocinnamide (SB-366791); 2,3,4-Trihydroxy-6-methyl-5-[(2E,6E)-3,7,11-trimethyl-2,6,10-dodecatrienyl]benzaldehyde (Scutigeral); 6,7-Deepoxy-6,7-didehydro-5-deoxy-21-dephenyl-21-(phenylmethyl)-20-(4-hydroxybenzeneacetate)daphnetoxin (Tinyatoxin); capsaicin synthetics; capsaicin derivatives; botulinum toxin; anti-convulsants; anesthetics; analgesics; opioids; cannabinoids; N-[2-(4-Chlorophenyl)ethyl]-1,3,4,5-tetrahydro-7,8-dihydroxy-2H-2-benzazepine-2-carbothioamide (Capsazepine); [N-(4-Hydroxy-3-methoxyphenyl)methyl]-5Z,8Z,11Z,14Z-eicosatetraenamide] (Arvanil); N-(3-Methoxyphenyl)-4-chlorocinnamide (SB-366791); 51-iodoresiniferatoxin; anti-inflammatory agents; steroids; nonsteroidal anti-inflammatory compounds; COX inhibitors; modulators of TNF-alpha or IL-1 cytokines or receptors; NFkB modulators; minocyclin and fluorocitrate.

16. The method of claim 14 wherein the biologically active agent is attached to the hygroscopic compound.

17. The method of claim 15 wherein the biologically active agent is selected from a set consisting of natural or synthetic neurotoxins comprising ammonia or cyanide; bisbenzimide; trypan blue; brilliant blue; methylene blue; indocyanine green; ruthenium red; quinoline yellow; saporin; Rho kinase activators; camphor; menthol; piperine; mustard oil; eugenol; curcumin; 8-Methyl-N-vanillyl-trans-6-nonenamide (Capsaicin); Z-Capsaicin; Gingerol; Zingerone; 8-Methyl-N-vanillylnonanamide (Dihydrocapsaicin); 6,7-Deepoxy-6,7-didehydro-5-deoxy-21-dephenyl-21-(phenylmethyl)-daphnetoxin,20-(4-hydroxy-5-iodo-3-methoxybenzeneacetate) (5′-Iodoresiniferatoxin); (+)-Isovelleral; N-Vannilyloleoylamide (Olvanil); Phorbol 12,13-dinonanoate 20-homovanillate; Resiniferatoxin; N-(3-Methoxyphenyl)-4-chlorocinnamide (SB-366791); 2,3,4-Trihydroxy-6-methyl-5-[(2E,6E)-3,7,11-trimethyl-2,6,10-dodecatrienyl]benzaldehyde (Scutigeral); 6,7-Deepoxy-6,7-didehydro-5-deoxy-21-dephenyl-21-(phenylmethyl)-20-(4-hydroxybenzeneacetate)daphnetoxin (Tinyatoxin); capsaicin synthetics; capsaicin derivatives; botulinum toxin; anti-convulsants; anesthetics; analgesics; opioids; cannabinoids; N-[2-(4-Chlorophenyl)ethyl]-1,3,4,5-tetrahydro-7,8-dihydroxy-2H-2-benzazepine-2-carbothioamide (Capsazepine); [N-(4-Hydroxy-3-methoxyphenyl)methyl]-5Z,8Z,11Z,14Z-eicosatetraenamide] (Arvanil); N-(3-Methoxyphenyl)-4-chlorocinnamide (SB-366791); 5′-iodoresiniferatoxin; steroids; nonsteroidal anti-inflammatory compounds; COX inhibitors; modulators of TNF-alpha or IL-1 cytokines or receptors; NFkB modulators; minocyclin and fluorocitrate.

18. The method of claim 14 wherein the biologically active agent is an anti-inflammatory agent.

19. The method of claim 1 wherein the hygroscopic compound is selected from a set consisting of poly (ethylene glycol), a block copolymer containing a polyalkylene glycol, triblock containing a polyalkylene glycol, a block copolymer containing a polyalkylene oxide, triblock containing a polyalkylene oxide, polyvinyl alcohol, polyvinyl pyrrolidone, dextrans, poloxamine, pluronic polyols, dimethylsulfoxide, starch, hydroxyethylstarch, sodium carboxymethyl cellulose, poly(polyethylene glycol methacryalte), poly(glycerol methacrylate), poly(glycerol acrylatete), poly(polyethylene glycol acrylate), poly(alkyl oxazoline), phosphoryl choline polymers, sodium and potassium polymethacrylate, sodium and potassium polyacrylate, polymethacrylatic acid and polyacrylic acid, glycerol, glycerin, sugars, hyaluronic acid, collagen, and proteoglycans.

20. The method of claim 1 wherein the hygroscopic agent further comprises an imaging agent.

21. A kit comprising:
a hygroscopic agent comprising a hygroscopic compound; and
instructions for administering the hygroscopic agent to a non-ruptured disc.

22. The kit of claim 21 further comprising a biologically active agent.

23. The kit of claim 21 further comprising an imaging agent.
